(19) United States
(12) Patent Application Publication
(10) Pub. No.: US 2008/0269556 A1
(43) Pub. Date: Oct. 30, 2008

(54) ENDOSCOPE WITH FLEXIBLE TIP

(76) Inventors: Ashok A. Jagasia, Naperville, IL (US); Jonathan Pomerantz, Chicago, IL (US)

Correspondence Address: FOLEY & LARDNER LLP, 150 EAST GILMAN STREET, P.O. BOX 1497, MADISON, WI, 53701-1497, US

(21) Appl. No.: 12/060,607

(22) Filed: Apr. 1, 2008

Related U.S. Application Data
(60) Provisional application No. 60/909,623, filed on Apr. 2, 2007.

(51) Int. Cl. A61B 1/01; A61B 1/018

(52) U.S. Cl. 600/104; 600/118

(57) ABSTRACT

An endoscope is provided. The endoscope includes a support body; a shaft mounted to the support body; a micro motor mounted in the support body; a flexible tip mounted to the shaft; and a directional cable. The directional cable is operably coupled to the micromotor at a first end and to the flexible tip at a second end and extends through the shaft. The flexible tip is configured for movement to a plurality of positions relative to the shaft through movement of the directional cable under control of the micro motor.

### CROSS-REFERENCE TO RELATED APPLICATIONS

This application claims the benefit of U.S. Provisional Patent Application Ser. No. 60/909,623 that was filed Apr. 2, 2007, the disclosure of which is incorporated herein by reference in its entirety.

### FIELD

The subject of the disclosure relates generally to endoscopes. More specifically, the disclosure relates to an endoscope which includes a flexible tip which is capable of being moved to a plurality of positions relative to a shaft of the endoscope.

### BACKGROUND

Endoscopic sinus surgery (also called endoscopy or sinoscopy) is a procedure used to remove blockages in the sinuses. If left in the sinus passages, these blockages can cause sinusitis, a condition in which the sinuses swell and become clogged, causing pain and impaired breathing. Endoscopic sinus surgery is generally performed with an instrument called an endoscope which allows a doctor to access and to inspect an interior of the nasal cavity. The endoscope includes a light source used to illuminate the nasal cavity, and a viewing panel through which the doctor can view the nasal cavity. Using the endoscope, the doctor can locate blockages such that the blockages can be removed with a scalpel, biopsy forceps, or other medical tool.

A traditional endoscope for use in endoscopic sinus surgery includes a rigid tip statically mounted to a rigid shaft. The rigid tip is often mounted to the rigid shaft such that the rigid tip and the rigid shaft form a static angle. The static angle can be any angle, but is generally zero degrees, thirty degrees, forty-five degrees, or seventy degrees. Unfortunately, the nasal passage, which is neither linear nor uniform, cannot be adequately examined from a single angle. Thus, doctors must utilize a plurality of endoscopes during endoscopic sinus surgery to fully examine the entire nasal passage. For example, the doctor may use a first endoscope in which the rigid tip is mounted at an angle of zero degrees to inspect a first portion of a nasal passage, a second endoscope in which the rigid tip is mounted at an angle of thirty degrees to inspect a second portion of the nasal passage, and a third endoscope in which the rigid tip is mounted at an angle of forty-five degrees to inspect a third portion of the nasal passage.

Using a plurality of endoscopes to conduct a single endoscopic sinus surgery results in a lengthy surgery because the doctor has to continually switch back and forth between endoscopes to fully inspect the nasal passage. Lengthy surgeries require a large amount of the doctor's valuable time and also cause prolonged discomfort to the patient. Lengthy surgeries also increase the risk that the patient will develop an infection, and continually inserting and removing different endoscopes into the nasal passage increases the risk that the patient's nasal passage will be damaged. In addition, endoscopes are expensive medical devices, and the need to have a plurality of distinct endoscopes to complete a single surgery is expensive for the doctor and/or their employer.

Thus, there is a need for an endoscope which includes a flexible tip such that the endoscope is capable of being used to view a nasal passage from a plurality of angles.

### SUMMARY

An exemplary endoscope includes a shaft and a flexible tip mounted to the shaft. The flexible tip includes a flexible body and a tip end. The tip end includes a digital lens or other reflecting mechanism such that a user of the endoscope can view an interior of a body part. The flexible body can bend relative to the shaft such that the tip end of the flexible tip can face a plurality of different directions about the shaft. The flexible tip can also be adjusted such that the flexible body can be placed at a plurality of different angles relative to the shaft. The flexible tip can be controlled by one or more direction cables which are mounted to the flexible tip and to one or more motors within a control box of the endoscope. The user can control the one or more motors through a control panel in electrical communication with the one or more motors.

In an exemplary embodiment, an endoscope is provided. The endoscope includes a support body; a shaft mounted to the support body; a micro motor mounted in the support body; a flexible tip mounted to the shaft; and a directional cable. The directional cable is operably coupled to the micromotor at a first end and to the flexible tip at a second end and extends through the shaft. The flexible tip is configured for movement to a plurality of positions relative to the shaft through movement of the directional cable under control of the micro motor.

In another exemplary embodiment, an endoscope is provided. The endoscope includes a support body; a shaft mounted to the support body; a flexible tip mounted to the shaft; a first micromotor mounted in the support body; a second micromotor mounted in the support body; a first directional cable operably coupled to the first micromotor at a first end and to the flexible tip at a second end, the first directional cable extending through the shaft; and a second directional cable operably coupled to the second micromotor at a first end and to the flexible tip at a second end, the second directional cable extending through the shaft. The flexible tip is configured for movement to a plurality of positions relative to the shaft through movement of the first directional cable under control of the first micromotor and of the second directional cable under control of the second micromotor

Other principal features and advantages will become apparent to those skilled in the art upon review of the following drawings, the detailed description, and the appended claims.

### BRIEF DESCRIPTION OF THE DRAWINGS

Exemplary embodiments will hereafter be described with reference to the accompanying drawings.

FIG. 1 is an endoscope with a flexible tip in accordance with an exemplary embodiment.

FIG. 2 is a partial view of an exemplary endoscope in which the flexible tip has been adjusted in an upward direction such that a flexible body of the flexible tip and the shaft form an angle of approximately ninety degrees.

FIG. 3 is a diagram illustrating a front view of a flexible tip and degrees of freedom thereof in accordance with an exemplary embodiment.

FIG. 4 is a cross-sectional view of the shaft in accordance with an exemplary embodiment.

FIG. 5 is a cutaway view of a control box of the endoscope in accordance with an exemplary embodiment.

FIG. 6A is a partial view of direction cables running through the shaft and the flexible tip in accordance with an exemplary embodiment.

FIG. 6B is a rear view of direction cable mounting points of the flexible tip in accordance with an exemplary embodiment.

### DETAILED DESCRIPTION

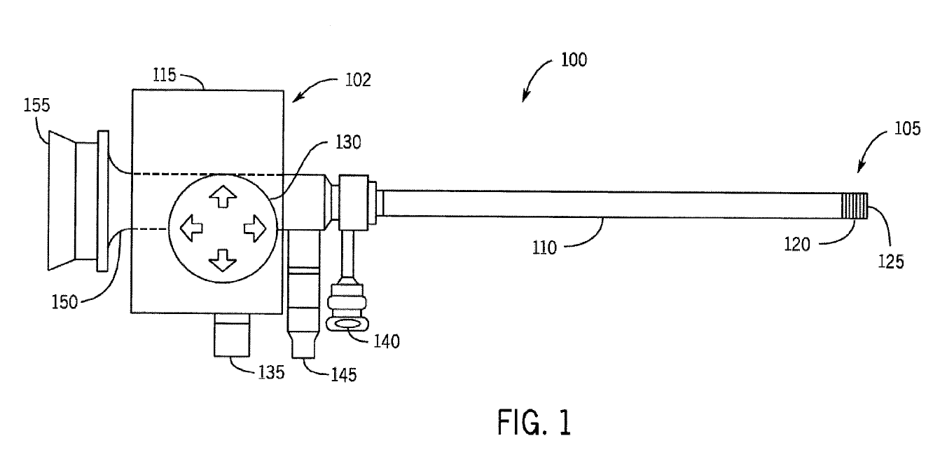

FIG. 1 provides an endoscope 100 that includes, but is not limited to, a support body 102, a flexible tip 105, and a shaft 110 in accordance with an exemplary embodiment. Flexible tip 105 is mounted to shaft 110. As used herein, the term ‘mount’ can include join, unite, connect, associate, insert, hang, hold, affix, attach, fasten, bind, paste, secure, bolt, nail, glue, screw, rivet, solder, weld, and other like terms. In an exemplary embodiment, shaft 110 is a rigid shaft which is approximately seventeen centimeters in length. In an exemplary embodiment, shaft 110 is formed of a hard plastic. Alternatively, shaft 110 may be flexible and/or shaft 110 may be any other length. In an exemplary embodiment, flexible tip 105 can be approximately one centimeter in length though other lengths can be used.

Flexible tip 105 includes a flexible body 120 and a tip end 125. In an exemplary embodiment, flexible body 120 is formed in a manner described in U.S. Pat. No. 6,991,603 incorporated herein by reference in its entirety. Flexible tip 105 can be adjusted such that flexible body 125 and shaft 110 form any of a plurality of angles between −180 degrees and 180 degrees. As illustrated with reference to FIG. 1, flexible tip 105 is positioned such that flexible body 125 and shaft 110 form an angle of zero degrees. As such, tip end 125 is positioned in a plane which is substantially perpendicular to shaft 110. In an exemplary embodiment, flexible tip 105 can have two degrees of freedom relative to the position illustrated with reference to FIG. 1, a pitch degree of freedom and a yaw degree of freedom.

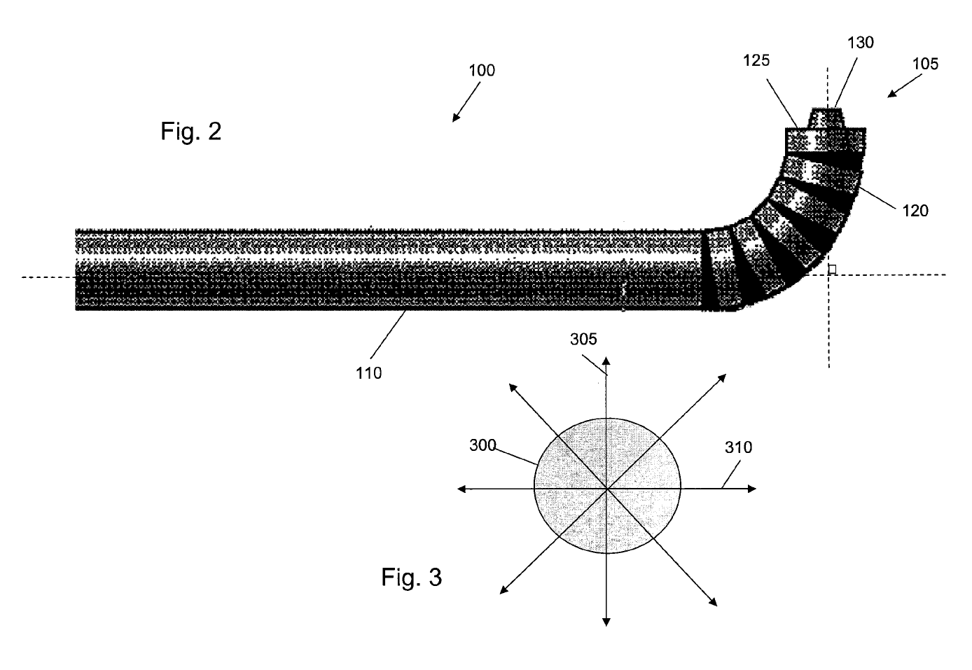

FIG. 2 provides a partial view of endoscope 100 in which flexible tip 105 has been adjusted in an upward pitch direction such that flexible body 120 and shaft 110 form an angle of approximately ninety degrees. As such, tip end 125 lies in a plane, which is substantially parallel to shaft 110. In an exemplary embodiment, flexible tip 105 can be adjusted to move from −180 degrees and 180 degrees in the pitch direction and from −180 degrees and 180 degrees in the yaw direction. The user can also rotate the endoscope, which is supported in the user's hand.

FIG. 3 is a diagram illustrating a front view of a flexible tip 300 and degrees of freedom thereof in accordance with an exemplary embodiment. A flexible body (not shown) of flexible tip 300 extends from a shaft (not shown) to which flexible tip 300 is mounted. As illustrated with reference to FIG. 3, flexible tip 300 can be moved in a pitch direction 305 as illustrated with reference to flexible tip 105 in FIG. 2. Flexible tip 300 can also be moved in a yaw direction 310. Flexible tip 300 can be moved in both pitch direction 305 and yaw direction 310 to provide positioning anywhere in the plane defined by pitch direction 305 and yaw direction 310 and from zero degrees (pointing out of the page) to ninety degrees (lying in the plane).

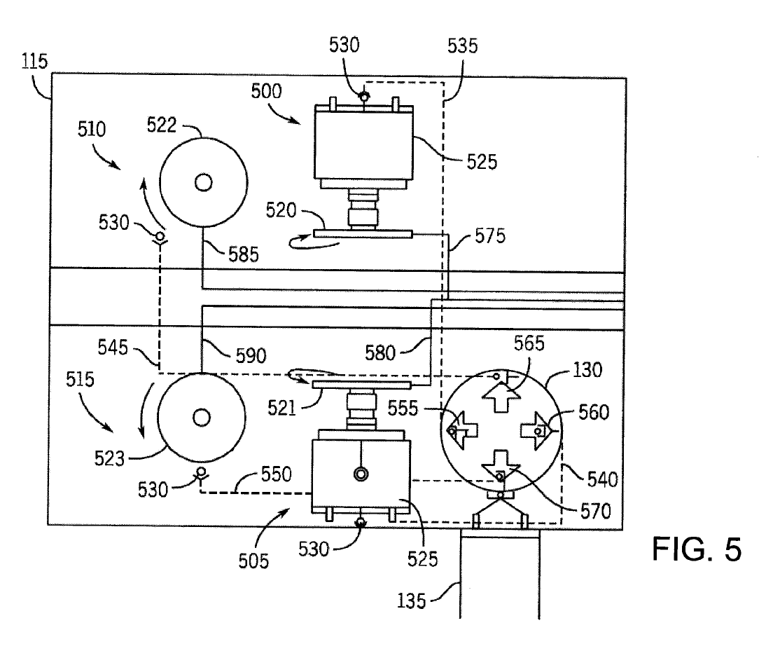

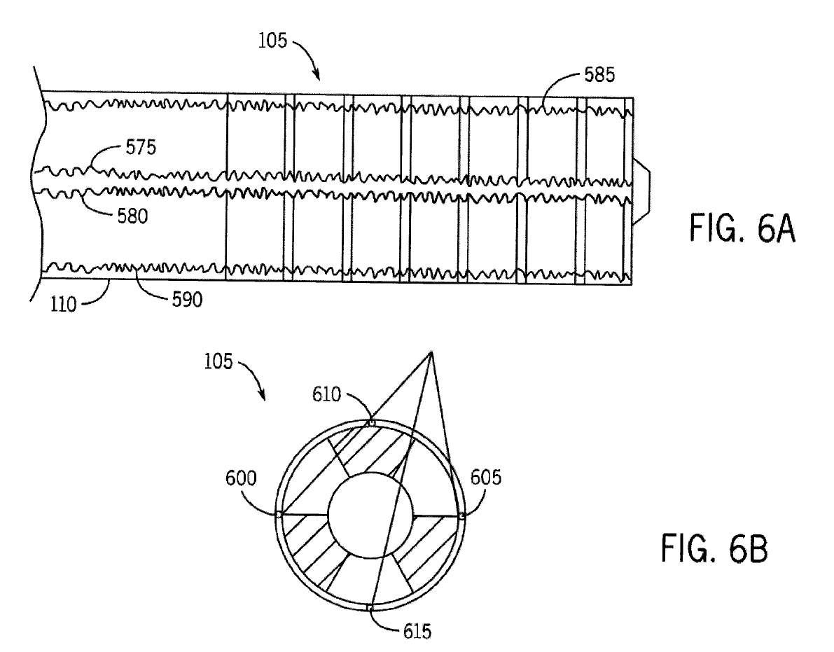

Referring back to FIG. 1, support body 102 includes a control box 115, a power input 135, a tool port 140, a light port 145, and a viewing port 150 to which shaft 110 is operably coupled. Control box 115 can house one or more motors capable of automatically adjusting a position of flexible tip 105. Motors and their use to control flexible tip 105 are described in more detail with reference to FIGS. 5, 6A, and 6B. A control panel 130 can be in communication with control box 115 such that a user of endoscope 100 can adjust flexible tip 105. In an exemplary embodiment, control panel 130 can be a key pad which includes buttons corresponding to up, down, left, and right. Alternatively, control panel 130 can be a toggle, joystick, digital control, trackball, or any other device through which the user can adjust the positioning of flexible tip 105.

Power input 135 provides power to control box 115. In an exemplary embodiment, power input 135 can be an electrical cord and plug capable of being plugged into a conventional power outlet. Alternatively, power input 135 can be a battery connector, a generator connector, or any other mechanism capable of connecting endoscope 100 to a power source. The power source (not shown) can be a conventional power outlet, a battery, a generator, or any other source capable of providing an electrical current to endoscope 100.

In an exemplary embodiment, tool port 140 can be in communication with a tool channel which runs through at least a portion of shaft 110 and at least a portion of flexible tip 105. Tool port 140 can receive any tool capable of being used in conjunction with endoscope 100. In an exemplary embodiment, tool port 140 can receive a suction catheter which is mounted to a vacuum or other suction device. In an exemplary embodiment, tool port 140 has a 2 millimeter diameter. The suction catheter can run through the tool channel such that blockage, mucus, blood, and any other bodily materials can be removed from a cavity or organ into which endoscope 100 is inserted. In another exemplary embodiment, a suction catheter 130 can extend beyond tip end 125 of flexible tip 105 as illustrated with reference to FIG. 2. Alternatively, the suction catheter can be flush with tip end 125. In another alternative embodiment, the tool channel can be the suction catheter and tool port 140 can be mounted directly to the suction device. Tool port 140 can also receive biopsy forceps which can be used by the user to remove blockage in a nasal cavity, or any other bodily materials in an organ, cavity, etc. The biopsy forceps can extend through the tool channel and out of tip end 125 such that the user can use the biopsy forceps simultaneously with endoscope 100. Alternatively, tool port can receive any other surgical tools or instruments, including a scalpel, a laser, etc. In one embodiment, tool port 140 can be capable of simultaneously receiving a plurality of tools which can be used with endoscope 100. In an alternative embodiment, endoscope 100 can include a plurality of tool ports, each of which can be capable of receiving a surgical tool.

Light port 145 can be mounted to a light source (not shown) such that a body part (i.e., cavity, organ, etc.) into which endoscope 100 is inserted can be illuminated. The light source can be any light source known to those of skill in the art. Light port 145 can be in communication with one or more light channels which run through at least a portion of shaft 110 and at least a portion of flexible tip 105. As such, light from the light source can travel through light port 145, through the light channel(s), and illuminate the body part into which endoscope 100 is placed. The light can further travel through one or more viewing channels which are in communication with viewing port 150 such that the user can see what they are doing within the body part.

The one or more viewing channels can run through at least a portion of shaft 110 and at least a portion of flexible tip 105. Any or all of the one or more viewing channels can also include a digital camera lens, a non-digital camera lens, any other lens, a mirror, or any other surface capable of reflecting light to viewing port 150 such that the user can see the body part within which the endoscope is located. A camera 155 can be mounted to viewing port 150 such that the user can view and or capture images of the body part. Camera 155 can be a digital camera, a non-digital camera, a camcorder, or any other image capturing device known to those of skill in the art. In an alternative embodiment, camera 155 may not be included, and viewing port 150 can include an eyepiece, screen, or display such that the user can view the body part.

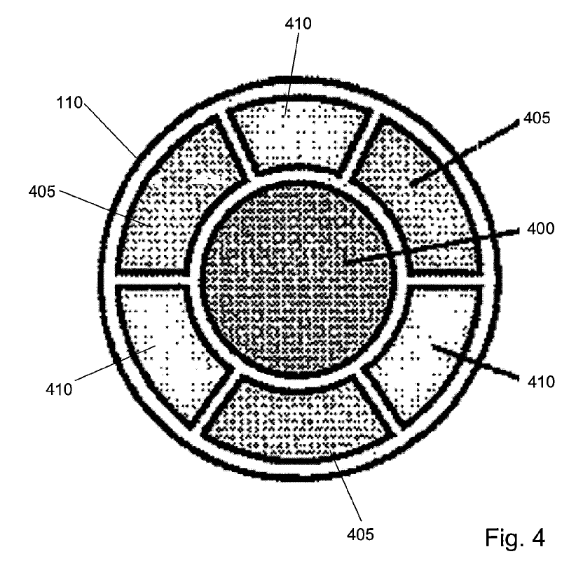

FIG. 4 is a cross-sectional view of shaft 110 in accordance with an exemplary embodiment. Shaft 110 includes a tool channel 400 capable of housing any surgical tool capable of being used with endoscope 100. Tool channel 400 can be in communication with tool port 140 described with reference to FIG. 1. In an alternative embodiment, shaft 110 may include a plurality of tool channels. Shaft 110 also includes a plurality of viewing channels 405 in communication with viewing port 150 described with reference to FIG. 1. In an alternative embodiment, shaft 110 may include a single viewing channel, or any other number of viewing channels, including two, four, five, etc. Shaft 110 further includes a plurality of light channels 410 in communication with light port 145 described with reference to FIG. 1. In an alternative embodiment, shaft 110 may include a single light channel, or any other number of light channels, including two, four, five, etc. In an exemplary embodiment, FIG. 4 can also be a cross-sectional view of flexible tip 105 described with reference to FIG. 1. In an exemplary embodiment, flexible tip 105 has a 4 millimeter diameter.

FIG. 5 is a cutaway view of control box 115 in accordance with an exemplary embodiment. Control box 115 includes a first motor 500, a second motor 505, a third motor 510, and a fourth motor 515 for use in adjusting flexible tip 105 described with reference to FIG. 1. In an exemplary embodiment, any or all of the motors can be micromotors. Alternatively, any or all of the motors can be nanomotors or any other type of motors known to those of skill in the art. In an exemplary embodiment, first motor 500 can be used to move flexible tip 105 in a leftward direction of yaw direction 310 described with reference to FIG. 3, second motor 505 can be used to move flexible tip 105 in a rightward direction of yaw direction 310, third motor 510 can be used to move flexible tip 105 in an upward direction of pitch direction 305, and fourth motor 515 can be used to move flexible tip 105 in a downward direction of pitch direction 305. The motors 500, 505, 510, 515 may be oriented in different directions and placed in different locations than those shown in FIG. 5 which is non-limiting.

In an exemplary embodiment, first motor 500, second motor 505, third motor 510, and fourth motor 515 can include a wheel (520, 521, 522, and 523, respectively), a motor body 525, and a power connector 530. Power connector 530 can be any connector capable of receiving power through a electrical lines 535, 540, 545, 550 in communication with control panel 130 described with reference to FIG. 1. Electrical line 535 can be used to provide a signal to first motor 500 such that first motor causes flexible tip 105 to move in a leftward direction. Power can be supplied to first motor 500 when a user presses a leftward direction button 555 on control panel 130. Electrical line 540 can be used to provide a signal to second motor 505 when the user presses a rightward direction button 560 on control panel. Similarly, electrical line 545 can be used to provide a signal to third motor 510 when the user presses an upward direction button 565 on control panel 130, and electrical line 550 can be used to provide a signal to fourth motor 515 when the user presses a downward direction button 570 on control panel 130. In an alternative embodiment, control panel 130 may not include directional buttons, and the user can provide power to the motors by moving a toggle, touching a digital screen, moving a joystick, etc.

In an exemplary embodiment, supplying power to first motor 500 can cause wheel 520 of first motor 500 to rotate. A leftward direction cable 575 is mounted to wheel 520 and to flexible tip 105 such that when wheel 520 rotates, flexible tip 105 moves in a leftward direction due to movement of leftward direction cable 575 about wheel 520. Similarly, a rightward direction cable 580 is mounted to wheel 521 of second motor 505 and to flexible tip 105 such that when wheel 521 rotates, flexible tip 105 moves in a rightward direction. An upward direction cable 585 is mounted to wheel 522 of third motor 510 and to flexible tip 105 such that when wheel 522 rotates, flexible tip 105 moves in an upward direction. A downward direction cable 590 is mounted to wheel 523 of fourth motor 515 and to flexible tip 105 such that when wheel 523 rotates, flexible tip 105 moves in a downward direction. In alternative embodiments, a fewer or a greater number of motors and or direction cables may be used.

In one embodiment, wheels 520, 521, 522, and 523 may rotate in a single direction as indicated by the arrows in FIG. 5. In addition, wheels 520, 521, 522, and 523 may all rotate in the same direction, or in different directions depending on the embodiment. Alternatively, any or all of wheels 520, 521, 522, and 523 may be able to rotate in either direction. In another embodiment, first motor 500 and second motor 505 can be oriented such that wheel 520 of first motor 500 faces wheel 521 of second motor 505. Third motor 510 and fourth motor 515 can be oriented in a side-by-side fashion such that third motor 510 is rotated approximately ninety degrees relative to first motor 500 and fourth motor 515 is rotated approximately ninety degrees relative to second motor 505. Alternatively, the motors can be oriented in any other manner such that flexible tip 105 can be adjusted.

In an exemplary embodiment, leftward direction cable 575, rightward direction cable 580, upward direction cable 585, and downward direction cable 590 can be housed within and run through at least a portion of shaft 110 described with reference to FIG. 1. FIG. 6A is a partial view of direction cables running through shaft 110 and flexible tip 105 in accordance with an exemplary embodiment. FIG. 6B is a rear view of direction cable mounting points of flexible tip 105 in accordance with an exemplary embodiment. In an exemplary embodiment, leftward direction cable 575 can be mounted to direction cable mounting point 600, rightward direction cable 580 can be mounted to direction cable mounting point 605, upward direction cable 585 can be mounted to direction cable mounting point 610, and downward direction cable 590 can be mounted to direction cable mounting point 615.

Further, for the purposes of this disclosure and unless otherwise specified, “a” or “an” means “one or more.”

The foregoing description of exemplary embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. An endoscope comprising:
a support body;
a shaft mounted to the support body;
a flexible tip mounted to the shaft;
a micromotor mounted in the support body; and
a directional cable operably coupled to the micromotor at a first end and to the flexible tip at a second end, the directional cable extending through the shaft;
wherein the flexible tip is configured for movement to a plurality of positions relative to the shaft through movement of the directional cable under control of the micromotor.

2. The endoscope of claim 1, wherein the shaft is formed of a rigid material.

3. The endoscope of claim 1, further comprising a plurality of channels, wherein the support body comprises a plurality of ports, a channel of the plurality of channels extending from a port of the plurality of ports to the flexible tip.

4. The endoscope of claim 3, wherein the plurality of ports include a light port.

5. The endoscope of claim 4, wherein the plurality of ports include a viewing port.

6. The endoscope of claim 5, wherein the plurality of ports include a tool port.

7. The endoscope of claim 3, wherein the plurality of channels extend through the shaft.

8. The endoscope of claim 3, wherein a center channel of the plurality of channels is configured to accept a tool.

9. The endoscope of claim 8, wherein the tool is selected from the group consisting of a suction catheter, a forceps, and a scalpel.

10. The endoscope of claim 1, further comprising a control panel in electrical communication with the micromotor to provide a control signal to the micro motor.

11. The endoscope of claim 10, wherein the directional cable is mounted to a wheel of the micromotor so that rotation of the wheel causes movement of the directional cable, and further wherein the micromotor controls rotation of the wheel based on the provided control signal.

12. The endoscope of claim 11, further comprising:
a second micromotor mounted in the support body; and
a second directional cable operably coupled to the second micromotor at a first end and to the flexible tip at a second end, the second directional cable extending through the shaft;
wherein the second directional cable is mounted to a second wheel of the second micromotor so that rotation of the second wheel causes movement of the second directional cable, and further wherein the second micromotor controls rotation of the second wheel based on the provided control signal.

13. The endoscope of claim 12, wherein the second end of the directional cable is positioned so that rotation of the wheel causes a first movement of the flexible tip in a first plane, and further wherein the second end of the second directional cable is positioned so that rotation of the second wheel causes a second movement of the flexible tip in a second plane.

14. The endoscope of claim 13, wherein the first plane and the second plane are the same plane.

15. The endoscope of claim 14, wherein the wheel of the first micromotor and the second wheel of the second micromotor are mounted to rotate in planes that are parallel to each other.

16. The endoscope of claim 13, wherein the first plane and the second plane are perpendicular to each other.

17. The endoscope of claim 16, wherein the wheel of the first micromotor and the second wheel of the second micromotor are mounted to rotate in planes that are perpendicular to each other.

18. An endoscope comprising:
a support body;
a shaft mounted to the support body;
a flexible tip mounted to the shaft;
a first micromotor mounted in the support body;
a second micromotor mounted in the support body;
a first directional cable operably coupled to the first micromotor at a first end and to the flexible tip at a second end, the first directional cable extending through the shaft; and
a second directional cable operably coupled to the second micromotor at a first end and to the flexible tip at a second end, the second directional cable extending through the shaft;
wherein the flexible tip is configured for movement to a plurality of positions relative to the shaft through movement of the first directional cable under control of the first micromotor and of the second directional cable under control of the second micromotor.

19. The endoscope of claim 18, wherein:
the first directional cable is mounted to a first wheel of the first micromotor so that rotation of the first wheel causes movement of the first directional cable, and further wherein the first micromotor controls rotation of the first wheel;
the second directional cable is mounted to a second wheel of the second micromotor so that rotation of the second wheel causes movement of the second directional cable, and further wherein the second micromotor controls rotation of the second wheel;
the second end of the directional cable is positioned so that rotation of the wheel causes a first movement of the flexible tip in a first plane, and
the second end of the second directional cable is positioned so that rotation of the second wheel causes a second movement of the flexible tip in a second plane.

20. The endoscope of claim 19, wherein the first wheel of the first micromotor is mounted to rotate in a plane that is parallel to the first plane.
